(19) United States
(12) Patent Application Publication
(10) Pub. No.: US 2019/0290155 A1
(43) Pub. Date: Sep. 26, 2019

(54) MEDICAL DEVICE AND METHOD FOR EVALUATING DATA FOR DEFECTS IN AN ELECTRODE LEAD

(71) Applicant: BIOTRONIK SE & CO. KG, BERLIN (DE)

(72) Inventors: RENE FISCHER, BERLIN (DE); ULRICH BUSCH, BERLIN (DE); SABRINA BAUDITZ, BERLIN (DE); ANDREAS NEUMANN, BERLIN (DE); PETER WOHLGEMUTH, CHEMNITZ (DE)

(21) Appl. No.: 16/361,575

(22) Filed: Mar. 22, 2019

(30) Foreign Application Priority Data
Mar. 23, 2018 (EP) 18163555.8

(51) Int. Cl. A61B 5/0424; A61N 1/04; A61N 1/08

(52) U.S. Cl. CPC A61B 5/0424; A61B 5/04087; A61N 1/08; A61N 1/0456

(57) ABSTRACT

A medical device has at least one electrode lead with at least one electrode pole that is configured to measure electrical potentials in human or animal tissue, and a measurement and control unit that is connected to the electrode lead. The measurement and control unit is configured to initiate measurements of the impedance via the electrode pole of the electrode lead. The measurements of the impedance have at least one individual measurement, an individual measurement occurring over a defined window of time.

### CROSS-REFERENCE TO RELATED APPLICATION

This application claims the priority, under 35 U.S.C. § 119, of European application EP 18163555.8, filed Mar. 23, 2018; the prior application is herewith incorporated by reference in its entirety.

### BACKGROUND OF THE INVENTION

### Field of the Invention

The invention describes a medical device and a method for evaluating data for defects in an electrode lead or between electrode leads or between electrode conductors and the housing.

Medical devices having electrode conductors for measuring electric potentials and for delivering electrotherapy have been known for decades. Such devices normally have a device housing in which are housed energy supply, energy storage unit, electronic components, etc. The electrode leads are coupled to the electronics in the device housing and lead out of the device housing.

One example of non-implantable medical devices are external electrocardiography monitoring devices (ECGs) (“Holter monitors”), which typically comprises an ECG measuring device and a plurality of electrode leads connected to the device. Disposed at the end of each electrode lead is a measuring electrode that is attached to an appropriate measurement point on this skin of the patient by an adhesive patch. The measurement signal recorded, which corresponds to the curve of the electrical potential from one measuring electrode to a second measuring electrode or to a neutral electrode, is in general called the “derivation.” Such a Holter monitor measures and stores a plurality of derivations simultaneously, which are subsequently provided to the doctor for analysis.

Transcutaneous electrical nerve stimulation (TENS) devices for external transcutaneous electrostimulation of nerve tissue are used to treat pain and stimulate muscles. They are another example of non-implantable medical devices having electrode leads: as with Holter monitors, TENS electrode leads have patch electrodes at their distal end. These patch electrodes are attached to the skin of the patient in the region to be stimulated. As a rule, low-frequency current stimulation pulses are emitted via the device.

Implantable devices are, for example, stimulation devices for the central nervous system, such as spinal cord stimulators (SCS), brain stimulation devices and vagus nerve stimulators (VNS). Cardiac stimulation systems and cardiac pacemaker systems have also been known for some time. There are cardiac stimulation systems for one chamber of the heart or for a plurality of chambers of the heart, depending on the need or pathophysiology of the patient. There are also cardiac stimulation systems with shock functions (implantable cardioverter defibrillators (ICDs)) that are able to administer a high-energy electric shock intracardially in order to treat life-threatening ventricular fibrillation.

Electrode leads for implantable systems normally have a longitudinally extended lead body that comprises a plurality of electrical conductors that are provided with electrical insulation. For example, the electrical conductors are covered with an insulating material such as silicone or plastic. Disposed at the proximal end of the electrode lead are contacts via which an electrical connection to the components in the device housing may be established. Electrode poles for receiving an electrical potential (e.g. for measuring physiological nerve activity) and/or for administering electrical stimulation therapy are disposed at the distal end of the electrode lead and/or on the lead body. Electrode poles may be embodied in different ways, for example, as tip electrodes or ring electrodes. There are also electrode poles in the form of shock coils; these are used in ICDs, for example, for providing the intracardial defibrillation shock.

As a rule, a plurality of electrical conductors is guided within one electrode lead. The electrical conductors are implemented, for example, in the form of wires or coils in order to ensure bendability and flexibility.

Due to continuous mechanical stressing of electrode leads, defects may occur in the insulation and in the electrical leads that may lead to derivation current, short circuits, elevated impedances, intermittent contacts, and/or breaks in the leads. Like breaks in the leads, contacting problems at terminals of an electrode lead for the stimulation device may occur. Such defects have a negative effect on the measurement and electrotherapy functions of the electrode lead. In some cases, a defect in the insulation or in the electrode lead leads to a short-circuit between two electrode leads or between an electrode lead and the housing of the stimulation device. In other cases, due to contact with tissue at the defective site, such a defect leads to voltages that overlay the actual physiological measurement signal for the patient and lead to erroneous detections by the medical device (e.g., to so-called “oversensing,” detection of false positive events). If this happens in a high-frequency disturbance in the measurement signal, an ICD may interpret this as cardiac tachycardia. If the device does not detect that the signal was caused by a defect in the insulation or lead, and not by actual patient tachycardia, this may result in inadequate administration of therapy. An inadequate defibrillation shock may be provided, which is extremely painful for the patient and should be avoided at all costs. Lead interruptions may lead to fatal failures in therapy, and in principle there is also the risk that the ICD will be damaged if shock energy is emitted in a defective electrode system.

Medical devices are known that are equipped with electronic systems and algorithms for detecting defects in the insulation or in electrical conductors of an electrode lead. For example, the electrical impedance between an electrode and the device housing is measured. Devices are known that measure a single impedance value of a derivation via such an impedance measurement. Individual impedance values are distributed over a time period (e.g. over a day) in order to calculate the mean from these individual values. If this mean moves within a certain range, the derivation examined is considered normal according to the impedance criterion.

U.S. Pat. No. 7,047,083 B2 describes a method in which an impedance value for an electrode lead is recorded at regular intervals. A short-term trend and a long-term trend are determined for the recorded impedance values. The trends are compared in order to determine the condition of the electrode lead.

U.S. Pat. No. 8,099,166 B2 describes an implantable cardiac device that examines an ECG to see if a threshold is exceeded. If the number of times a threshold is exceeded goes beyond a limit, a snapshot of the ECG is stored.

Typically, the electrode system mounted on a pacemaker or ICD is measured for impedance a few times during the day. If these measurements (which are frequently computed, for example, by averaging) are within a certain range, the examined lead is considered normal with respect to the impedance criterion.

However, the systems known to this point in time suffer from the drawback that certain electrode defects cannot be detected.

In particular in the case of defects that cause a characteristic measurement signal only intermittently, or defects in the initial formation, cannot be detected using known detection algorithms. Such defects are currently detected only by coincidence, frequently only once the patient has already suffered harm. Detection using known algorithms is not possible in particular when such defects occur in electrode leads that are not used to measure any physiological signal. Nor can such defects be detected by means of the known methods for monitoring impedance. They record individual impedance values for a derivation at defined times, so that the points in time at which the defect is manifest in the impedance signal are missed.

### SUMMARY OF THE INVENTION

One object of the present invention is to provide a method and a device for detecting certain scenarios for defects in an electrode or electrode lead, or defects in the insulation of an electrode lead. Embodiments of the invention are intended to detect so-called intermittent defects.

The present invention is intended for improved detection of defects in an electrode or electrode lead or defects in the insulation of an electrode lead. One object of the present invention is to provide a device and a method for preventing inadequate therapies due to erroneously interpreted sensing

The current consumption of a medical device should only be minimally limited by the use of the inventive method. For this, the invention provides suitable parameters and parameter range so that a high probability of detecting the aforesaid defects, with low current consumption, is made possible.

Another object of the invention is to discover breaks in leads and loose header screws and thus to assure it is possible to administer effective therapies.

One object of the invention is to provide a novel testing criterion for evaluating defects in an electrode or electrode lead or in the insulation.

The aforesaid objects are attained using a medical device according to the independent claim.

According to the present invention, a medical device is described that comprises:
a) at least one electrode lead having at least one electrode pole, the electrode lead being designed to measure electrical potentials in human or animal tissue, and
b) a measurement and control unit that is connected to the electrode lead, the measurement and control unit being configured to initiate measurements of the impedance via the electrode pole of the electrode lead or via the electrode poles of a plurality of electrode leads, characterized in that the measurements of the impedance has at least one individual measurement, and one individual measurement occurs over a defined window of time.

According to one embodiment of the invention, the measurement of the impedance may occur continuously, i.e. the window of time is not limited in duration.

According to one embodiment of the invention, the measurement of the impedance may have a plurality of individual measurements.

According to one aspect of the present invention, the window of time is 5 seconds to 360 seconds in length. According to one preferred embodiment of the present invention, the window of time is preferably approximately 90 seconds in length.

In one special embodiment of the invention, the window of time is 90 seconds in length.

According to one embodiment of the present invention, the individual measurements of impedance are samples by a sampling frequency, hereinafter called semi-continuous measurements. Alternatively, the individual measurements may also be continuous impedance measurements.

In one preferred embodiment of the invention, each individual measurement comprises a recording of impedance values by means of a sampling frequency, the sampling frequency being between 8 Hz and 128 Hz. According to one advantageous embodiment of the present invention, each individual measurement comprises a recording of impedance values by use of a sampling frequency of approximately 32 Hz.

In one special embodiment, each individual measurement comprises a recording of impedance values by use of a sampling frequency of 32 Hz.

According to one advantageous realization of the invention, there is defined time period of 0.5 hours to 24 hours between two individual measurements. In one preferred embodiment of the invention, there is a defined time period of approximately 1 hour between two individual measurements.

In one exemplary embodiment, all individual measurements of an overall measurement are evaluated jointly using the measurement and control unit.

According to one aspect of the present invention, an overall measurement preferably has a defined number of individual measurements. An overall measurement preferably takes place over a fixed time period, e.g. 12 hours, 24 hours, 48 hours. The time period should be selected such that patient safety is adequately provided for, and such that at the same time the battery run time is not significantly reduced by the evaluation.

According to one aspect of the invention, the measurement and control unit is preferably designed to seamlessly begin a new overall measurement upon completion of an overall measurement. In this way continuous measurement coverage is provided.

In one special embodiment, an overall measurement comprises a time period of 24 hours, an individual measurement comprises a window of time of 90 seconds, with a sampling frequency of 32 Hz.

The time period for an overall measurement, the window of time for an individual measurement, and the sampling frequency may be selected such that a high probability of discovering an intermittent electrode defect, with low current consumption by the medical device, is assured.

According to one embodiment of the invention, the measurement and control unit is configured to evaluate the measured impedance values. The evaluation looks for a defect in the electrode lead or in a plurality of electrode leads or for contact problems in a plug-in connector.

Alternatively, the medical device may be configured to provide the measured impedance values to an external device or to an external service center, where they are evaluated.

According to one embodiment of the invention, the measured impedances are evaluated in that the impedance values or differential values between the impedance values are compared to a threshold value. The threshold value is a predefined value, or may be updated regularly using current impedance measurements. According to one embodiment of the invention, the medical device is configured to transmit the stored impedance values and/or the differential values to an external device or to an external service center.

According to one aspect of the invention, the measurement and control unit evaluates measured impedance values in that differential values between the impedance values are calculated and the differential values are compared to a threshold value

In embodiments, the threshold value is in a range of 20 Ohms to 140 Ohms.

According to one embodiment of the invention, a differential value is preferably calculated between two discrete, successive impedance values.

According to one aspect of the present invention, the electrode pole is preferably associated with:

a) a right ventricular shock coil, or
b) an atrial shock coil, or
c) a ring electrode, or
d) a tip electrode of the electrode lead.

According to one aspect of the invention, the medical device comprises a device housing, wherein the device housing comprises an electrical pole or at least one part of the housing forms an electrical pole. The measurement and control unit is connected to the electrode pole and the electrical pole of the device housing. Moreover, the measurement and control unit is configured to conduct measurements of the impedance between the electrode pole and the electrical pole of the device housing.

According to one embodiment of the invention, the medical device has more than one electrode lead. At least one electrode lead may have more than one electrode pole. According to one aspect of the invention, the measurement and control unit is configured to conduct measurements of the impedance between two poles, wherein the two poles comprise at least a combination of electrode poles and/or the electrical pole of the device housing.

According to one embodiment of the inventive medical device, impedance values are evaluated individually by the measurement and control unit for each combination of electrode poles and/or the electrical pole of the device housing.

According to one preferred embodiment of the invention, the measurement and control unit is configured to increment a counter value for an individual measurement when a threshold value is exceeded by the calculated differential value and to store the measured impedance values when a counter limit is exceeded.

According to one embodiment of the present invention, the measurement and control unit is preferably designed to store impedance values of an individual measurement from a series of those individual measurements that represents the individual measurement from the series of individual measurements that has the highest counter value. Alternatively, the impedance values of an individual measurement that represents the chronologically first individual measurement from the series of individual measurements for which the counter limit was exceeded are stored. According to one embodiment, when stored, the impedance values are stored within a sub-window of time. The sub-window of time is preferably linked to the time at which the counter limit was exceeded.

According to one aspect of the present invention, the medical device is designed to transmit the stored impedance values to an external device or to an external service center.

According to one aspect of the present invention, the medical device is embodied as an implantable stimulation device for neurostimulation, such as e.g. for spinal cord stimulation, or vagus nerve simulation, or for a cardiac stimulation device, such as e.g. a cardiac pacemaker, an ICD, or a CRT device.

According to one embodiment of the present invention in which the medical device is embodied as an ICD, the measurement and control unit is configured to conduct measurements of the impedance between every two poles, the two poles comprising at least a combination of the following:

a) Right ventricular coil electrode and device housing,
b) Right ventricular coil electrode and right ventricular tip electrode,
c) Right ventricular ring electrode and device housing,
d) Right ventricular ring electrode and right ventricular tip electrode,
e) SVC coil electrode and device housing, and/or
f) SVC coil electrode and right ventricular coil electrode.

Also described is a method for evaluating impedance values for defects in an electrode lead. The method comprises the steps of:

a) measuring an impedance using the electrode pole of an electrode lead,
b) evaluating measured impedance values for defects in the electrode lead, and
c) characterized in that the measurements of the impedance has at least one individual measurement, and one individual measurement occurs across a defined window of time.

According to one embodiment of the inventive method, the measurement of the impedance may occur continuously, i.e., the window of time is not limited in duration.

According to one embodiment of the inventive method, the measurement of the impedance may have a plurality of individual measurements.

Within the scope of the invention, different embodiments and aspects of the inventive device are also applicable to the corresponding method for evaluating impedance values for defects in an electrode lead.

According to one embodiment of the present invention, impedance measurements should be conducted on the relevant measurement leads at least over one time period per day. For each individual measurement, the impedance is measured over a predefined window of time, wherein the samples of the sampled impedance signal are stored. According to one aspect of the invention, these measurements are evaluated in the medical device and/or in an external device. A counter is incremented if during the evaluation a discrepancy is found that is not consistent with the expected physiologically typical impedance curve. If the medical device is a cardiac implant, such as e.g. an ICD, the physiologically typical impedance curve is the type of impedance curve caused by the movement of the heart.

According to one embodiment of the present invention, the counter value of the incremented counter is evaluated after a predefined time. Thus the condition of a defect in the electrode lead and/or in the insulation may be detected when the counter value exceeds a counter limit.

According to one aspect of the present invention, once a defect has been detected, a corresponding follow-on measure is introduced by the medical device and/or the external device. For example, an alarm to an external device and/or to an external data center may be set by the medical device. If the medical device has a plurality of electrode poles for which detection of defects is conducted individually according to the invention, and if a defect is discovered in connection with an electrode (or derivation), the device may be configured such that the current path is automatically switched. For example, it may be switched to an electrode that is arranged physically adjacent to the one in which the defect was detected. Alternatively, the electrode in question may be temporarily deactivated.

If the medical device is an ICD, for example, and if a defect is detected in connection with an electrode pole with a sensing function, the device can automatically switch to another derivation for the measurement. If a defect is detected in connection with a shock coil, the device can automatically change the shock path. In this way it is possible to prevent a defect in the electrode lead (or on the electrode or in the insulation of the electrode) that has just formed from providing inadequate therapies (caused by defective sensing) or to prevent omission of a required shock (due to a defect in the electrode lead having a shock function).

The present invention offers a number of advantages. For one thing, reliable monitoring of the functionality of electrode leads for intermittently occurring defects is possible without the active involvement of the doctor. In connection with the initiation of an automated notification/alarm when a defect is detected, e.g. on an external device or an external data center, a process for reliable early notification of such defects are implemented.

The present invention also offers a high level of sensitivity and specificity (i.e., avoids false positive alarms) for detecting the aforesaid defect scenarios. The inventive device and method permits reliable differentiation between impedance signal curves produced by the aforesaid defects and those that are produced by physiological events in the patient, such as e.g. movement of the body.

According to one embodiment of the present invention, impedance measurements should be conducted on the relevant measurement leads at least over one time period per day. For each individual measurement, the impedance is measured over a predefined window of time, the impedance signal being stored by means of a sampling frequency by sample. According to one aspect of the invention, these measurements are evaluated in the medical device and/or in an external device. A counter is incremented if during the evaluation a discrepancy is found that is not consistent with the expected, physiologically typical impedance curve. If the medical device is a cardiac implant, such as e.g. an ICD, the physiologically typical impedance curve is type of impedance curve that would be caused by the movement of the heart.

According to one embodiment of the present invention, the counter value of the incremented counter is evaluated after a predefined period. Thus the condition of a defect in the electrode lead and/or in the insulation may be detected if the counter value exceeds a counter limit.

According to one aspect of the present invention, once a defect has been detected, a corresponding follow-on measure is introduced by the medical device and/or the external device. For example, an alarm to an external device and/or an external data center set. If the medical device has a plurality of electrode poles for which detection of defects is conducted individually according to the invention, and if a defect is discovered in connection with an electrode (or derivation) is discovered, the device may be configured such that the current path is automatically switched. For example, it may be switched to an electrode that is arranged physically adjacent to the one in which the defect was detected. Alternatively, the electrode in question may be temporarily deactivated.

If the medical device is an ICD, for example, and if a defect is detected in connection with an electrode pole with a sensing function, the device may switch automatically to another derivation for the measurement. If a defect is detected for a shock coil, the device can automatically change the shock path. In this way it is possible to prevent a defect in the electrode lead (or on the electrode or in the insulation of the electrode) that has just formed from providing inadequate therapies (caused by defective sensing) or to prevent omission of a required shock (due to a defect in the electrode lead having a shock function).

The present invention offers a number of advantages. For one thing, reliable monitoring of the functionality of electrode leads for intermittently occurring defects is possible without the active involvement of the doctor. In the connection with initiation of an automated notification/alarm when a defect is detected, e.g. on an external device or an external data center, a process for reliable early notification of such defects are implemented.

The present invention also offers a high level of sensitivity and specificity for detecting the aforesaid defect scenarios. The inventive device and method permits reliable differentiation between impedance signal curves produced by the aforesaid defects and those that are produced by physiological events in the patient, such as e.g. movement of the body.

Other features which are considered as characteristic for the invention are set forth in the appended claims.

Although the invention is illustrated and described herein as embodied in a medical device and a method for evaluating data for defects in an electrode lead, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE SEVERAL VIEWS OF THE DRAWING

FIG. 1 is an illustration depicting an exemplary embodiment of the present invention using a stimulation device having a ring electrode and a tip electrode;

FIG. 2 is an illustration depicting an exemplary embodiment of the present invention using a stimulation device having a ring electrode, a tip electrode, and a shock coil; and

FIG. 3 is an illustration depicting an exemplary embodiment of the present invention using a stimulation device having a ring electrode, a tip electrode, and two shock coils.

### DETAILED DESCRIPTION OF THE INVENTION

Functionally equivalent or identically acting elements in the figures are provided the same reference numbers. The figures are schematic representations of the invention. They do not illustrate specific parameters of the invention. Moreover, the figures merely reflect typical embodiments of the invention and shall not limit the invention to the illustrated embodiments.

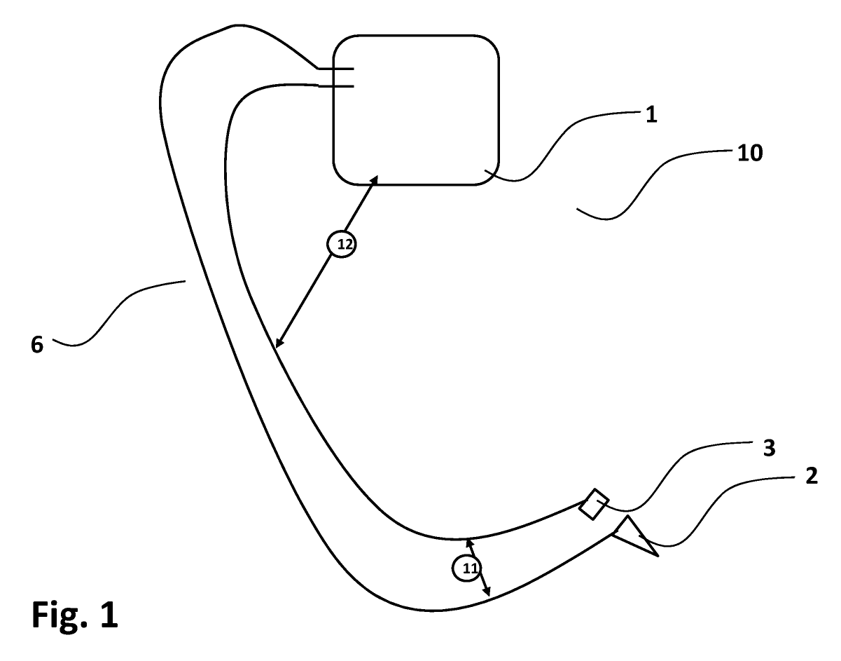

Referring now to the figures of the drawings in detail and first, particularly to FIG. 1 thereof, there is shown an exemplary embodiment of the present invention using a stimulation device 10 having a ring electrode 3 and a tip electrode 2. Specific embodiments are implantable cardiac pacemakers, brain pacemakers, spinal cord stimulators, and the like. The stimulation device 10 comprises a device housing 1, the tip electrode 2, the ring electrode 3, and electrical lines 6 for the electrodes. According to the invention, the impedance may be measured for detecting defects in the electrode, electrode conductors, or insulation, for example via

a) Derivation 11 (between tip electrode 2 and ring electrode 3) and/or
b) Derivation 12 (between ring electrode 3 and device housing 1).

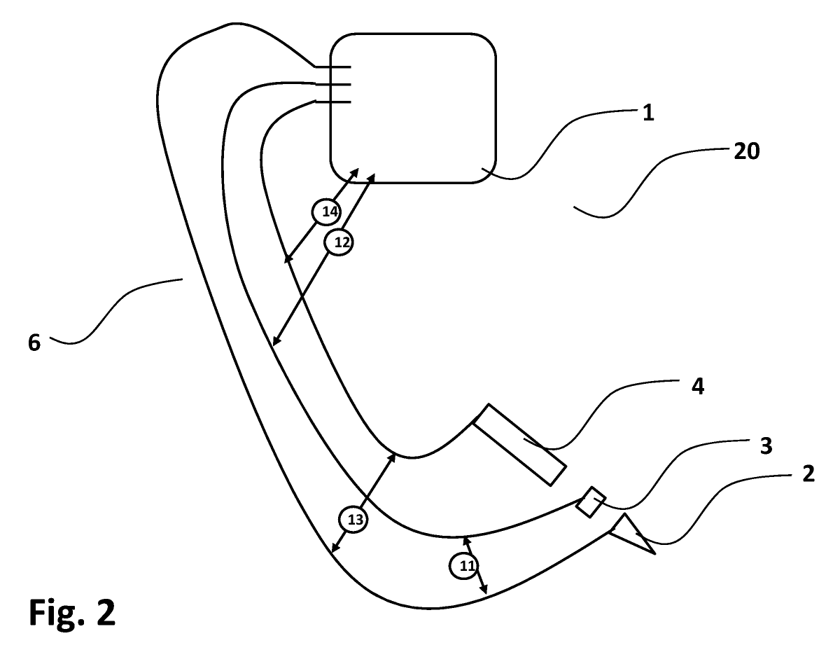

FIG. 2 depicts an exemplary embodiment of the present invention using a stimulation device 20 having the ring electrode 3, the tip electrode 2, and a shock coil 4. One specific embodiment is an implantable cardioverter defibrillator (ICD). The stimulation device 20 comprises the device housing 1, the tip electrode 2, the ring electrode 3, the shock coil 4, and the electrical leads 6 for the electrodes. According to the invention, the measurement of the impedance for detecting defects in the electrode, electrode lead, or insulation may be conducted, for instance, using:

a) Derivation 11 (between tip electrode 2 and ring electrode 3), and/or
b) Derivation 12 (between ring electrode 3 and device housing 1), and/or
c) Derivation 13 (between tip electrode and shock coil 4), and/or
d) Derivation 14 (between shock coil 4 and device housing 1).

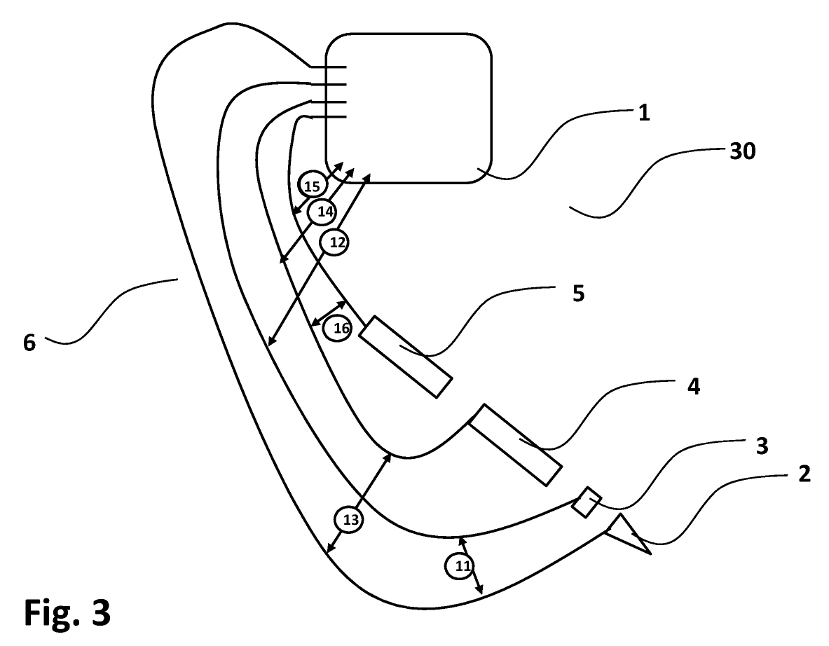

FIG. 3 depicts one exemplary embodiment of the present invention using a stimulation device 30 having a ring electrode, a tip electrode, a shock coil 4, and another shock coil 5. One specific embodiment is an implantable cardioverter defibrillator (ICD) having a right ventricular shock coil and an SVC shock coil. The stimulation device 30 comprises the device housing 1, the tip electrode 2, the ring electrode 3, the shock coil 4, shock coil 5, and the electrical lines 6 for the electrodes. According to the invention, the measurement of the impedance for detecting defects in the electrode, electrode lead, or insulation may be conducted, for example, using:

a) Derivation 11 (between tip electrode 2 and ring electrode 3), and/or
b) Derivation 12 (between ring electrode 3 and device housing 1), and/or
c) Derivation 13 (between tip electrode and shock coil 4), and/or
d) Derivation 14 (between shock coil 4 and device housing 1), and/or
e) Derivation 15 (between shock coil 5 and device housing 1), and/or
f) Derivation 16 (between shock coil 4 and shock coil 5).

According to one aspect of the invention, changes in impedance above a specific slew rate are detected and evaluated. According to one embodiment of the invention, a differential value between two discrete, successive impedance values is preferably calculated for the signal evaluation. According to one example, measurements of the impedance are conducted between every two electrical poles, the two poles comprising at least a combination of the following:

a) Right ventricular coil electrode and device housing,
b) Right ventricular coil electrode and right ventricular tip electrode,
c) Right ventricular ring electrode and device housing,
d) Right ventricular ring electrode and right ventricular tip electrode,
e) SVC coil electrode and device housing, and/or
f) SVC coil electrode and right ventricular coil electrode.

According to one preferred embodiment of the invention, the measurement and control unit is configured to increment a counter value for an individual measurement when a threshold value is exceeded by the calculated differential value, and to store the measured impedance values when a counter limit is exceeded.

According to one aspect of the invention, the aforesaid threshold value is a function of the observed derivation.

According to one embodiment in which the medical device is preferably embodied as an implantable cardiac therapy device, at least one counter for the specific derivation may be incremented when the impedance difference by sample is measured higher than:

a) Right ventricular coil electrode and device housing: approx. or exactly 24 Ohms,
b) Right ventricular coil electrode and right ventricular tip electrode: approx. or exactly 78 Ohms,
c) Right ventricular ring electrode and device housing: approx. or exactly 78 Ohms,
d) Right ventricular ring electrode and right ventricular tip electrode: approx. or exactly 137 Ohms,
e) SVC coil electrode and device housing: approx. or exactly 24 Ohms, and/or
f) SVC coil electrode and right ventricular coil electrode: approx. or exactly 40 Ohms.

According to one embodiment, the frequency of these events per time segment (e.g. day) is counted and evaluated.

According to one embodiment, alternatively or in addition to the above described embodiment, the absolute impedance may be compared, by sample, to an absolute threshold value. The absolute threshold value may be parameterized specifically for the electrode used or may be automatically continuously revised from the previous measurements (e.g. from trend data).

### REFERENCE SIGNS

1 Device housing
2 Tip electrode
3 Ring electrode
4 Shock coil
5 Shock coil
6 Electrode leads
10 Medical device
11 Derivation from tip electrode 2 to ring electrode 3
12 Derivation from ring electrode 3 to device housing 1
13 Derivation from tip electrode to shock coil 4
14 Derivation from shock coil 4 to device housing 1
15 Derivation from shock coil 5 to device housing 1
16 Derivation from shock coil 4 to shock coil 5
20 Medical device
30 Medical device

### ABBREVIATIONS AND DEFINITIONS OF TERMS

| | |
|---|---|
| Derivation/measurement | In the context of the invention, derivation and |
| derivation | measurement derivation shall be construed to mean a |
| | recorded measurement signal that corresponds to the |
| | course of the electrical potential from one measurement |
| | electrode to a second measurement electrode or to a |
| | neutral electrode. |
| Distal end | For an electrode lead, the end that is arranged farthest |
| | from the device housing. |
| ECG | Electrocardiograph |
| Electrode/electrode pole | In the context of the invention, electrode and electrode |
| | pole shall be construed to mean a metal unit connected |
| | to an electrode lead, the unit, in combination with a |
| | second electrode or a counterelectrode, permitting |
| | recording of voltage potentials or current to be output. |
| Electrode lead | In the context of the invention, an electrode lead shall |
| | be construed to mean a line made of electrically |
| | conducting material. Electrodes or electrode poles for |
| | measuring voltage potentials or for outputting current |
| | may disposed at the end of or along the electrode lead. |
| | As a rule, an electrode lead is formed from a plurality of |
| | electrode conductors that are insulated from one |
| | another. |
| ICD | Implantable cardioverter defibrillator |
| IEGM | Intrakardiales Elektrogram (English: intracardial |
| | electrogram) |
| Intermittent | In the context of the invention, “intermittent” shall be |
| | construed to describe a temporary and non-continuous |
| | event. |
| Insulation | Insulation shall be construed to be the means for |
| | electrically insulating an electrode lead. |
| Proximal end | For an electrode lead, the end that is coupled to the |
| | device housing. |
| Semi-continuous | In the context of the invention, “semi-continuous” |
| | describes a measurement that takes place continuously |
| | over a certain time period or over a certain window of |
| | time. In the case of measurements of impedance values |
| | that are detected by sample via a sample frequency, |
| | from a purely technical perspective this is not a |
| | “continuous” measurement (as would be the case for an |
| | analog measurement signal) - therefore the term “semi- |
| | continuous.” The invention shall not be limited to a |
| | semi-continuous measurement of impedance, however. |
| SCS | Spinal cord stimulation/spinal cord stimulator |
| Sensing | In the context of the invention, the term “sensing” shall |
| | be construed to mean the detection of physiological |
| | signals by measuring electrical potentials. |
| SVC | Superior Vena Cava |
| TENS | Transcutaneous electrical nerve stimulation |
| VNS | Vagus nerve stimulation |

1. A medical device, comprising:
at least one electrode lead having at least one electrode pole, said electrode lead configured to measure electrical potentials in human or animal tissue; and
a measurement and control unit connected to said electrode lead, said measurement and control unit configured to initiate measurements of impedance via said electrode pole of said electrode lead, the measurements of impedance have a plurality of individual measurements, and one individual measurement occurs over a defined window of time, and changes in impedance above a specific slew rate are detected and evaluated.

2. The medical device according to claim 1, wherein the defined window of time is 5 seconds to 360 seconds in length.

3. The medical device according to claim 1, wherein each of the individual measurements is a recording of impedance values by means of a sampling frequency, the sampling frequency being between 8 Hz and 128 Hz.

4. The medical device according to claim 1, wherein there being a defined time period of 0.5 hours to 24 hours between two individual measurements.

5. The medical device according to claim 4, wherein the defined time period is 1 hour between the individual measurements.

6. The medical device according to claim 1, wherein:
said measurement and control unit evaluates measured impedance values; or
the medical device is configured to provide the measured impedance values to an external device or to an external service center, where they are evaluated.

7. The medical device according to claim 6, wherein the measured impedance values being evaluated in that the measured impedance values or differential values between the measured impedance values are compared to a threshold value, the threshold value being a predefined value, or the threshold value being a value that is updated regularly using current impedance measurements, the medical device being configured to transmit stored impedance values and/or the differential values to the external device or to the external service center.

8. The medical device according to claim 7, wherein a differential value is calculated between two discrete, successive impedance values.

9. The medical device according to claim 1,
further comprising a right ventricular shock coil,
further comprising an atrial shock coil;
further comprising a ring electrode;
further comprising a tip electrode connected to said electrode lead;
wherein said electrode pole is associated with one of said right ventricular shock coil, said atrial shock coil, said ring electrode, or said tip electrode of said electrode lead;
further comprising a device housing having a further electrical pole;
wherein said measurement and control unit is connected to said electrode pole and said further electrical pole of said device housing; and
wherein said measurement and control unit is configured to conduct measurements of the impedance between said electrode pole and said further electrical pole of said device housing.

10. The medical device according to claim 9, wherein:
said electrode lead is one of a plurality of electrode leads; and/or
said electrode lead has a plurality of electrode poles; and
said measurement and control unit is configured to conduct a measurement of the impedance between two of said electrode poles, wherein said two electrode poles have at least a combination of said electrode poles and/or said further electrical pole of said device housing.

11. The medical device according to claim 10, wherein impedance values are evaluated individually for each combination of said electrode poles and/or said further electrical pole of said device housing.

12. The medical device according to claim 6, wherein said measurement and control unit is configured to increment a counter value for an individual measurement when a threshold value is exceeded by the measured impedance values or by a calculated differential value, and to store the measured impedance values when a counter limit is exceeded.

13. The medical device according to claim 12, wherein said measurement and control unit is configured to store the measured impedance values of an individual measurement from a series of individual measurements, the individual measurement represents the individual measurement from the series of individual measurements that has a highest counter value, or represents a chronologically first individual measurement from the series of individual measurements for which the counter limit was exceeded.

14. The medical device according to claim 12, wherein the measured impedance values are stored within a sub-window of time, the sub-window of time being linked to a time at which the counter limit is exceeded.

15. A method for evaluating impedance values for defects in an electrode lead, which comprises the steps of:
measuring an impedance using an electrode pole of the electrode lead; and
evaluating measured impedance values for defects in the electrode lead, wherein measurements of the impedance have at least one individual measurement, and the one individual measurement occurs across a defined window of time.
